(19) United States
(12) Patent Application Publication
(10) Pub. No.: US 2025/0384478 A1
(43) Pub. Date: Dec. 18, 2025

(54) SYSTEM AND METHOD FOR MATCHING CANNABIS USERS TO CANNABIS PRODUCTS

(71) Applicants: Luke Patterson, Hollywood, SC (US); Jacob Patterson, Hollywood, SC (US); Gary Heitz, Austin, TX (US); Nick Heldreth, La Jolla, CA (US)

(72) Inventors: Luke Patterson, Hollywood, SC (US); Jacob Patterson, Hollywood, SC (US); Gary Heitz, Austin, TX (US); Nick Heldreth, La Jolla, CA (US)

(21) Appl. No.: 19/172,479

(22) Filed: Apr. 7, 2025

Related U.S. Application Data
(63) Continuation-in-part of application No. 17/826,580, filed on May 27, 2022, now abandoned.

(51) Int. Cl. G06Q 30/0601

(52) U.S. Cl. CPC G06Q 30/06311

(57) ABSTRACT

A system for matching a cannabis user to a cannabis product is disclosed, including at least one user computing device in operable connection with a user network. An application server is in operable communication with the user network to host an application system for providing a system for providing a recommendation engine to match user information to a cannabis product which provides desired effects that match the user information. The application system includes a user interface module for providing access to the application system through the user computing device. The user interface module is in operable communication with a user database to store user information and a product database to store product information. A seed-to-sale profile includes provider information, plant genetics information, and reviews and is displayed via the user interface module. The recommendation engine receives the user information to match the user information to cannabis products.

### CROSS-REFERENCE TO RELATED APPLICATIONS

The present application is a continuation-in-part of and claims priority to U.S. Non-Provisional patent application Ser. No. 17/826,580 filed May 27, 2022, titled “AUXILIARY POWER SYSTEM FOR A SEMI-TRUCK,” which is hereby incorporated by reference in its entirety.

### TECHNICAL FIELD

The embodiments generally relate to computerized systems and methods for the categorization and recommendation of a regulated product and more specifically related to a recommendation engine for cannabis and associated products.

### BACKGROUND

Historically, retail purchasing by a consumer has been done at a “brick and mortar” store. This has the advantage of allowing the consumer to interact with a salesperson or product expert who can recommend products based on the consumers personal preferences, needs, and desired outcomes. Recently, advances and the general prevalence of online marketplaces have left consumers with task of researching a product, without the interaction with a product expert.

Recently, changes in regulations have allowed legal cannabis to be extensively cultivated throughout the world. A family of active compounds specific to cannabis provide a wide range of benefits and sought after effects. Specifically, cannabinoids have been identified to provide a wide range of effects and benefits to humans. Each cannabinoid varies in concentration depending on the strain of cannabis being cultivated, quality of the seed, grow technique, storage technique, among other variables. Common and well-known cannabinoids include delta-9-tetrahydrocannabinol (THC), cannabidiol (CBD), and cannabinol (CBN) which each provide unique effects and benefits to the user.

Another family of active compounds are terpenes which are a large family of aromatic branched hydrocarbons produced by many plant species. Different cannabis strains may produce widely different terpene profiles and their derivative, terpenoids. These active compounds give each cannabis strain a unique scent and flavor when consumed. It is also noted that some terpenes may provide effects similar to some cannabinoids, such as providing the sedative, euphoric, and pain-reducing properties often associated with THC.

It is the differences in the levels and types of cannabinoids, terpenes, and even flavonoids that impart cannabis strains with unique desirable effects, flavors, and scents. Users may have a set of preferred strains based on the effects, flavors, and scents which they desire. For example, users may prefer Cannabis indica strains or Cannabis sativa strains due to their unique properties. Each has key differences in size, density, odor, smoke flavor, effects. While many users are aware of this key difference, users are often left uneducated about the more nuanced differences between particular strain of each the thousands of strains of cannabis. Further, each cultivation of the same strain may vary in levels of cannabinoids, terpenes, and flavonoids resulting in inconsistent effects when consumed.

How the cannabis is consumed can drastically change the perceived effects. It is well known that cannabis can be smoked, vaporized, decarboxylated and ingested orally, applied topically, or be dissolved in a liquid to be consumed topically or orally. With the complex nature of the plant and its application, many users are not educated on the most suitable strain and method of consumption for their desired effects.

### SUMMARY OF THE INVENTION

This summary is provided to introduce a variety of concepts in a simplified form that is disclosed further in the detailed description of the embodiments. This summary is not intended to identify key or essential inventive concepts of the claimed subject matter, nor is it intended for determining the scope of the claimed subject matter.

The embodiments provided herein relate to a system for matching a cannabis user to a cannabis product is disclosed, including at least one user computing device in operable connection with a user network. An application server is in operable communication with the user network to host an application system for providing a system for providing a recommendation engine to match user information to a cannabis product which provides desired effects that match the user information. The application system includes a user interface module for providing access to the application system through the user computing device. The user interface module is in operable communication with a user database to store user information and a product database to store product information. A seed-to-sale profile includes provider information, plant genetics information, and reviews and is displayed via the user interface module. The recommendation engine receives the user information to match the user information to cannabis products.

The system can interpret laboratory results which provide analytics data (e.g., terpene profiles, CBD content and percentages, THC content and percentages (among other known cannabinoids), flavonoid content and percentages). Further, the system permits a user to input information such as user analytics, user type (e.g., recreational or medicinal), desired effects, and the like to suggest suitable strains, method of consumption, products type and where the product can be purchased.

In one aspect, the system includes a machine learning-based personalization engine that dynamically adjusts recommendations using federated learning across user devices.

In one aspect, the system includes a blockchain-backed seed-to-sale authentication module to ensure traceable and tamper-proof tracking of cannabinoid, terpene, and contaminated profiles.

In some aspects, the system includes a mapping system which applies neural networks trained on anonymized clinical datasets to predict likely user responses.

In some aspects, the system includes on-device secure processing where sensitive user health and biometric data are processed locally to ensure HIPAA-compliant privacy standards.

In one aspect, the system allows for monitoring and analysis of the seed-to-sale cycle. The system monitors the location-based sale-to-seed monitoring of the plant that the user will purchase, rather than strain data which is often not accurate across plant cultivars and growers. This allows the user to see each step of the growing, cultivation, storage, and distribution processes.

In one aspect, the system may utilize and artificial intelligence (AI) engine to recommend cannabis products based on the user's previous experience and interactions with cannabis products. In such, the system may determine the user's desired effects and cannabis strains or related products which are known to produce such effects based on the user-input data.

In one aspect, the plurality of user information is comprised of at least one of the following: at least one desired effect, at least one symptom, and at least one user type.

In one aspect, the plurality of provider information includes at least one of the following: a provider profile, a plurality of related results, a water regimen, and a nutrient regimen.

In one aspect, the system includes a plurality of plant genetics information includes at least one of the following: one or more parent plant genetics, at least one certificate of analysis, and one or more laboratory results.

In one aspect, the user information includes a plurality of user demographics including at least one of the following: a height, a weight, a sex/gender, an ethnicity/race, an experience level, one or more generated feedbacks, and a purchase history.

In one aspect, the product database includes a plurality of laboratory results to provide cannabinoid concentrations, a product type, a terpene profile, and a flavonoid profile.

In one aspect, the recommendation engine recommends a dosage protocol based on the user information.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a block diagram of a computing system utilized to operate the application program and recommendation engine described herein, according to some embodiments;

FIG. 2 illustrates a block diagram of a computing system and an application program, according to some embodiments;

FIG. 3 illustrates a block diagram of the application program and associated databases, according to some embodiments;

FIG. 4 illustrates a flowchart of a process for matching a cannabis user to a cannabis product, according to some embodiments;

FIG. 5 illustrates a block diagram of a process for matching a cannabis user to a cannabis product including the process for the user inputting user information, according to some embodiments;

FIG. 6 illustrates a block diagram of a process for matching a cannabis user to a cannabis product including the process of storing product information, according to some embodiments;

FIG. 7 illustrates a block diagram of a process for matching a cannabis user to a cannabis product including the process of storing laboratory results, according to some embodiments;

FIG. 8 illustrates a block diagram of a process for matching a cannabis user to a cannabis product including the patient including the process of storing patient demographics, according to some embodiments; and

FIG. 9 illustrates a block diagram of a process for matching a cannabis user to a cannabis product, according to some embodiments.

### DETAILED DESCRIPTION

The specific details of the single embodiment or variety of embodiments described herein are to the described system and methods of use. Any specific details of the embodiments are used for demonstration purposes only, and no unnecessary limitations or inferences are to be understood thereon.

Before describing in detail exemplary embodiments, it is noted that the embodiments reside primarily in combinations of components and procedures related to the system. Accordingly, the system components have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein.

In this disclosure, the various embodiments may be a system, method, and/or computer program product at any possible technical detail level of integration. A computer program product can include, among other things, a computer-readable storage medium having computer-readable program instructions thereon for causing a processor to carry out aspects of the present disclosure.

In general, the embodiments described herein relate to systems and methods for providing a recommendation engine to match cannabis users to cannabis products. The system can interpret laboratory results which provide analytics data (e.g., terpene profiles, CBD content and percentages, THC content and percentages (among other known cannabinoids), flavonoid content and percentages). Further, the system permits a user to input information such as user analytics, user type (e.g., recreational or medicinal), desired effects, and the like to suggest suitable strains, method of consumption, products type and where the product can be purchased.

In some embodiments, the system allows for monitoring and analysis of the seed-to-sale cycle. The system monitors the location-based sale-to-seed monitoring of the plant that the user will purchase, rather than strain data which is often not accurate across plant cultivars and growers.

In some embodiments, the system may utilize and artificial intelligence (AI) engine to recommend cannabis products based on the user's previous experience and interactions with cannabis products. In such, the system may determine the user's desired effects and cannabis strains or related products which are known to produce such effects based on the user-input data.

### 1. Machine Learning Personalization Engine

In some embodiments a machine learning personalization module is integrated with user computing devices that uses a recurrent neural network (RNN) to monitor behavioral inputs such as strain selection patterns, reported effectiveness, dosage timing, and even biometric feedback (e.g., sleep patterns via smartwatch integration). The engine is trained locally and syncs summary weights to a central model.

In some embodiments, the system provides real-time lab data integration. Using standardized COA (Certificate of Analysis) APIs, the system ingests lab data regarding cannabinoid profiles, contaminants, moisture content, and more. Data ingestion is timestamped and hashed using a distributed ledger technology (DLT) for integrity.

In some embodiments, the system provides phenotype-based prediction models using phenotype correlation engines, user genetic profiles (if available), demographic data, and historical feedback are analyzed to predict optimal strain-product combinations. This goes beyond preference and introduces a biological response modeling system.

Data is processed on-device whenever possible, including AI inference tasks, using secure enclaves (e.g., ARM TrustZone or Intel SGX). No raw personal data is transmitted unless explicitly authorized.

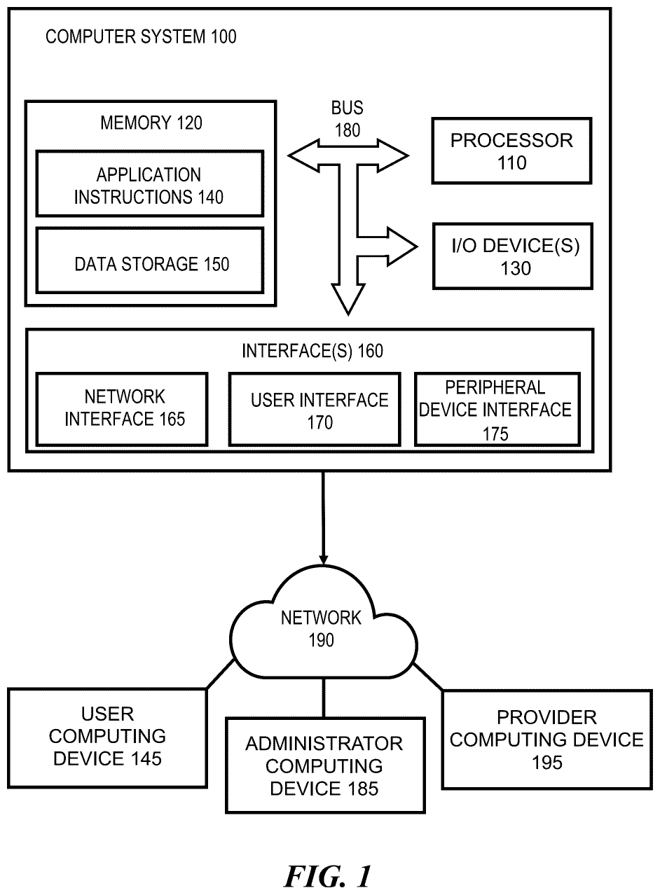

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computing device 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 150, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., C, C++, C #, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

As used herein, the term “symptom” or “symptoms” are sensations, conditions, or other physical or mental feature regarded as indicating a condition of a disease which is particularly apparent to the patient such as, for example, headaches, nausea, loss-of-appetite, seizures, muscle aches, muscle spasms, general or acute pain, etc. The user or patient may also input a desired relief similar to the input of a symptom.

Desired effects may include euphoria, increase in appetite, stimulation of creativity, aid in falling and/or staying asleep, pain management, calming sensations, happiness, sense of well-being, and other effects often associated with the consumption or application of cannabis and related products thereof.

As used herein, the term “history of use” which is input by the user, can include the users preferred strains, preferred method of administration (e.g., by smoking or vaporizing, ingesting, topically applying, etc.), preferred dosage amount (or a maximum/minimum dosage), previous providers, previous cultivators, and the like.

As used herein, the term “product(s)” and “cannabis product” may relate to any available presentation and preparation of cannabis known in the arts. This may include, but is not limited to, cannabis flower, strains, edibles, concentrates (e.g., preparations of wax, “shatter”, “budder”, oil(s), etc., topicals (e.g., lotion, balm, etc.), tinctures, beverages, pre-prepared cigars, and the like.

As used herein, the term “provider(s)” may be utilized to describe recreational and/or medicinal suppliers of cannabis products, cultivators, seed providers, and the like. In such, the providers may provide cannabis products to other providers and/or to users/patients.

As used herein, the term “user type” relates to the user's status as a recreational user and/or medical patient. In such, the system may then determine an appropriate provider to recommend to the user.

In some embodiments, the computer system 100 may include a user computing device 145, an administrator computing device 185 and a provider computing device 195 each in communication via the network 190. The user computing device 145 may be utilized by users of the system who are interested in searching for and/or purchasing a product from one or more providers. The user computing device 145 is operable to permit the user to input user information (e.g., preferences, symptoms, desired effects, history of use, and user type). The administrator computing device 185 is utilized by an administrative user who may manage user content, user permissions, input general product information, to transmit information and communicate with users, or otherwise interact with the system. The provider computing device 195 may be utilized by providers of cannabis products to input product information, update and maintain inventory, communicate with users, and otherwise interact with the various functionalities of the system.

The system's product recommendation schema may be based on objective data and can also be used in combination with user feedback about the subjective effects of products to power a recommendation engine which can be recommended products that will have similar or different effects based on whether they fall into the same or different product groupings and categories.

In another embodiment, a computer program product comprising machine executable instructions stored on non-transitory machine readable media is provided. The instructions provide for recommending a product for consumption by implementing a method of obtaining user input including at least one of personal data (e.g., height, weight, sex, age, heartrate, blood pressure, ethnicity/race, experience level, generated feedback, purchase history, etc.), preference data (e.g., saved history of “liking” or “not liking” a given product), and experience data (e.g., saved history of how a product made them feel). The system may obtain provider input including at least one of general data descriptive of the product, batch data descriptive of the product and user data descriptive of perceived effects of the product on the user. The system may also utilize chemical data obtained from testing laboratories (which is used to categorize and visualize individual products) with the preference data obtained from user inputs. Further, the system may receive imagery of the product to display the visual appearance thereof. This may be especially important for user's wanting cannabis flower.

As discussed herein, the term “user account” generally refers to an account maintained on behalf of a user to facilitate at least one of tracking of user data, selection and order of a regulated product. As discussed herein, the term “merchant account” generally refers to an account maintained on behalf of a merchant to facilitate evaluation of merchant operations, such as sales operations, orders placed with suppliers, inventory and other related information for user selection and acquisition of the selected regulated product.

The recommendation system may implement self-training algorithms such as artificial intelligence. For example, the recommendation system may implement a neural network. The neural network may accept, for example, personal data to correlate observed physiological effects of products with the physiology of the user. The correlation may be for the individual user, a segment of the user population, or the user population as a whole.

The recommendation system may implement user security, privileges, certificates, and other techniques to ensure integrity of the process and authenticity of the regulated products delivered to the user. The recommendation system may be operated with regard for privacy laws, such as HIPAA (i.e., the “Health Insurance Portability and Accountability Act”) which sets forth requirements for control of medically sensitive information.

The system may include advertising and may be used to sell other items such as related paraphernalia, medicaments, or other related products. Further, the system may be configured for facilitating routine or automatic ordering of a product or set of products as well as the delivery thereof.

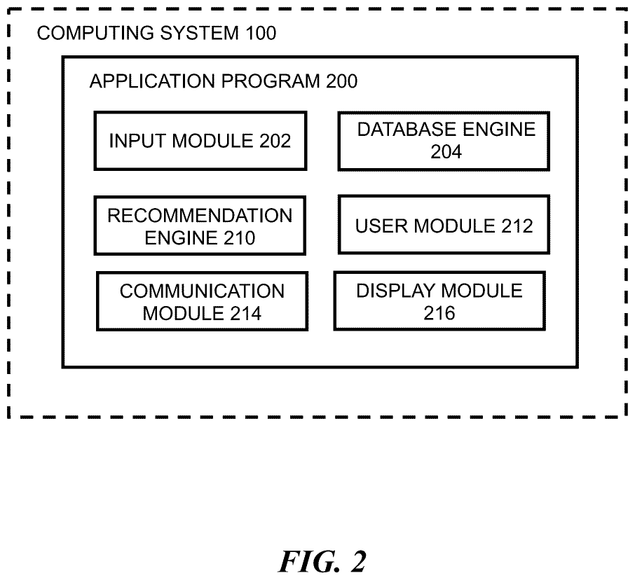

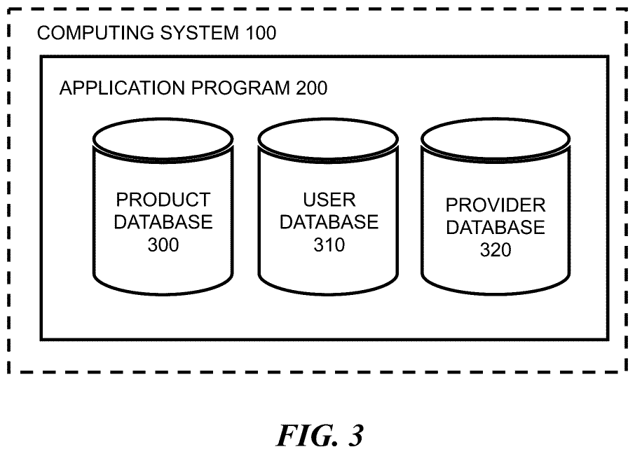

FIGS. 2 and 3 illustrate an example computer architecture for the application program 200 operated via the computing system 100. The computer system 100 comprises several modules and engines configured to execute the functionalities of the application program 200, and a database engine 204 configured to facilitate how data is stored and managed in one or more databases. In particular, FIG. 2 is a block diagram showing the modules and engines needed to perform specific tasks within the application program 200, and FIG. 3 is a block diagram showing the various databases utilized by the various modules.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of an input module 202, a database engine 204, a recommendation engine 210, a user module 212, a communication module 214, and a display module 216.

In some embodiments, the input module 202 permits a user to input user information which is stored in the user database 310. For example, the user may input information, via the user module 202, related to general contact information, location, desired effects, symptoms, desired relief, whether or not they are a recreational user or medicinal patient, history of use, and the like.

In some embodiments, the provider may input provider information, via the input module 202, such as provider information related to the provider's location, contact information, whether they service recreational users and/or medicinal patients, hours of operation, product inventory, product information, and the like.

In some embodiments, a database engine 204 is configured to facilitate the storage, management, and retrieval of data to and from one or more storage mediums, such as the one or more internal databases described herein. In some embodiments, the database engine 204 is coupled to an external storage system. In some embodiments, the database engine 204 is configured to apply changes to one or more databases. In some embodiments, the database engine 204 comprises a search engine component for searching through thousands of data sources stored in different locations. The database engine 204 may retrieve information from any database in communication with the system including the product database 300, user database 310, and provider database 320.

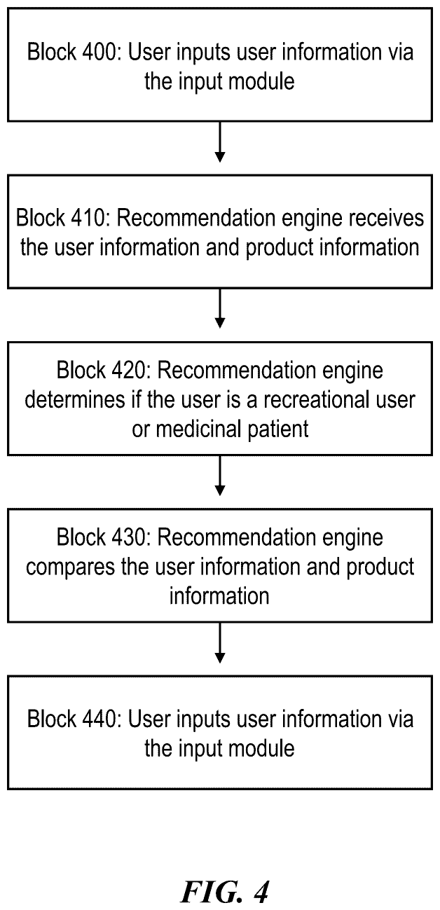

In some embodiments, the recommendation engine 210 receives user input as facilitated by the input module 202 and processes the information to provide one or more recommendations to the user. For example, the recommendation engine 210 interprets the user inputs of being a medicinal user who wants to purchase a product in the Miami area. Further, the user indicates they have insomnia and lack of appetite. The recommendation engine 210 may then suggest, via the processes described herein, one or more products and providers who sell the particular product(s) which match the user's needs. The recommendation engine 210 may utilize the processes described in the flowcharts included in FIGS. 4-8.

In some embodiments, a recommendation engine is provided which users can utilize in order to navigate products and product categories as well as determine a suitable product for their unique symptoms, desired relief, and desired effects.

In some embodiments, the user module 212 facilitates the creation of a user account and/or provider account for the application system. In such, the user account associates information input, via the input module 202, by the user or provider. The user module 212 may associate various permissions with each user as assigned by the administrative user.

In some embodiments, the communication module 214 is configured for receiving, processing, and transmitting a user command and/or one or more data streams. In such embodiments, the communication module 214 performs communication functions between various devices, including the user computing device 145, the administrator computing device 185, and a provider computing device 195. In some embodiments, the communication module 214 is configured to allow one or more users of the system, including a third-party (e.g., a delivery service), to communicate with one another. In some embodiments, the communications module 214 is configured to maintain one or more communication sessions with one or more servers, the administrator computing device 185, the provider computing device 195, and/or one or more user computing device(s) 195.

In some embodiments, the display module 216 is configured to display one or more graphic user interfaces, including, e.g., one or more user interfaces, one or more consumer interfaces, one or more video presenter interfaces, etc. In some embodiments, the display module 216 is configured to temporarily generate and display various pieces of information in response to one or more commands or operations. The various pieces of information or data generated and displayed may be transiently generated and displayed, and the displayed content in the display module 216 may be refreshed and replaced with different content upon the receipt of different commands or operations in some embodiments. In such embodiments, the various pieces of information generated and displayed in a display module 216 may not be persistently stored.

The display module 216 may be operable to display user information associated with the user's profile and account to the user, provider, and/or administrative user. In another example, the display module 216 displays provider information and product information to the user once the recommendation engine 210 has determined one or more suitable products and providers thereof. In another example, the display module 216 displays user/patient demographics to the provider. In such, the system includes a user interface for displaying individual products using a novel form of visualization preferably generated based on lab data quantifying the chemical profile of each product, as well as for receiving and assessing user-specific data relating to such products.

FIG. 3 illustrates a block diagram of the application program 200 operated via the computing system 100. The application program 200 comprises a product database 300, user database 310, and provider database 320. The product database 300 is configured to store product information including the seed-to-sale profile, grower information, plant genetics, user-generated feedbacks, user/patient demographics, lab results, and the like. The user database 310 stores user information such as the user's preferences, contact information, historical use data, and the like. The provider database 320 may store provider-associated information such as the providers contact information, inventory, locations, and the like.

FIG. 4 illustrates a flowchart for a process for matching a cannabis user to a cannabis product via the recommendation engine described hereinabove. In block 400, the user inputs user information via the input module which is stored in the user database. In block 410, the recommendation engine receives the user information and product information stored in the product database. In block 420, the recommendation engine determines if the user is a recreational user or a medicinal patient. In block 430, the recommendation engine compares the user information, including the users desired effects, symptoms, etc. and compares the user's preferences with the available products offered by providers in a specific region. The region may be determined from the user's location which they input in block 400. In block 440, a list of products generated by the user are displayed to allow the user to view product details, view provider details, etc.

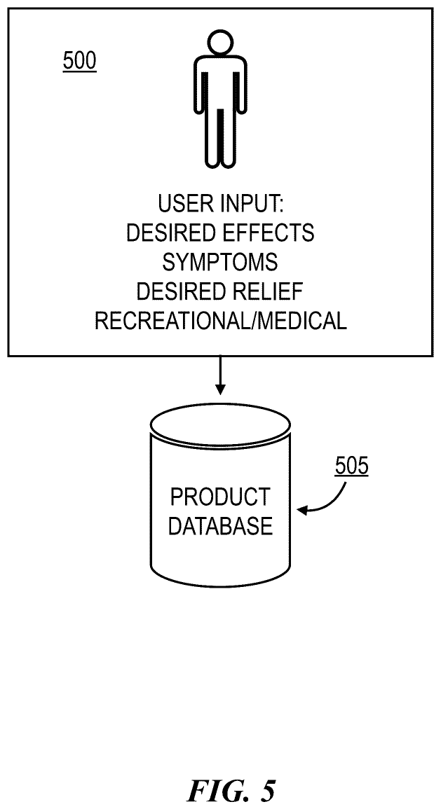

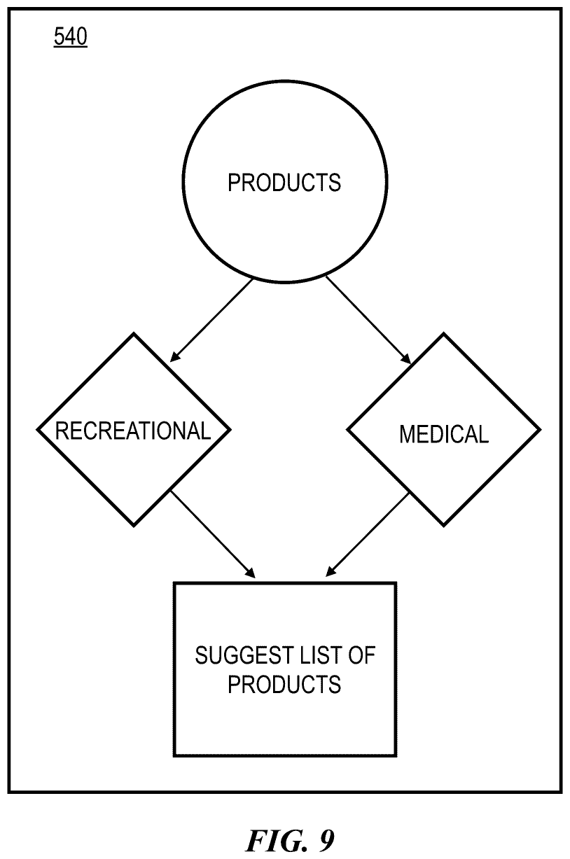

FIGS. 5-9 illustrate a flowchart of the process for recommending a cannabis product to a user via the system described herein above. FIG. 5 illustrates a flowchart, wherein in block 500, the user inputs user information including their desired effects, symptoms, desired relief, user type (i.e., whether they are a recreational user and/or a medical patient). The input module permits the input of such information which may be associated with the user's profile. The input module is operable by the application program provided on the computer system. In block 505, the input module transmits the user information to the user database and the product database. The product database stores the various strains, product types, edibles, concentrates, and topicals which are available.

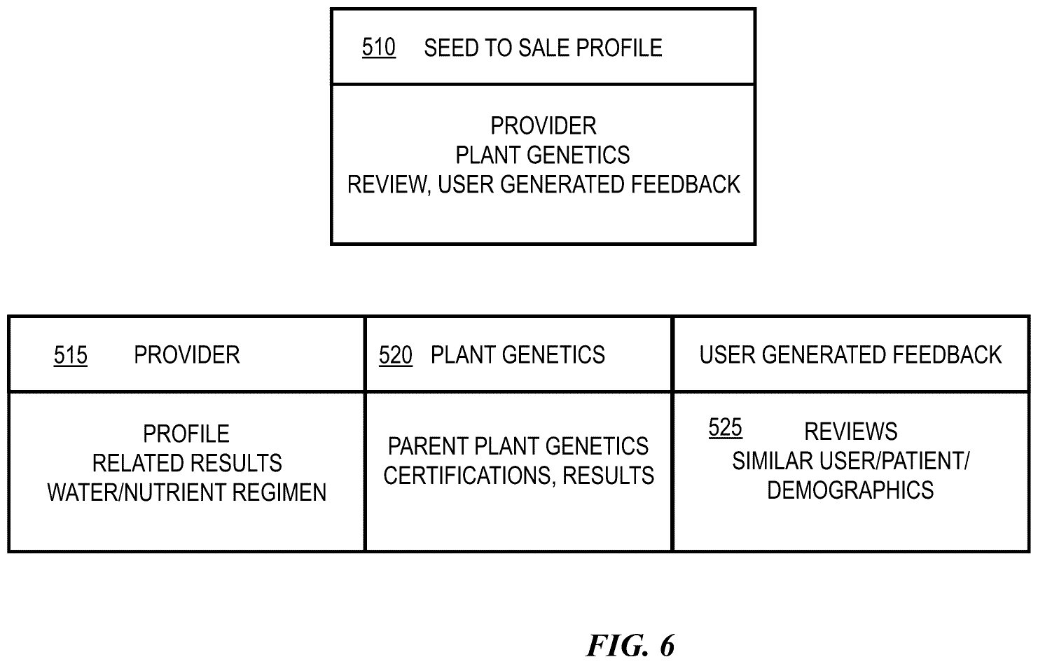

FIG. 6 illustrates a flowchart wherein the product information in the product database (see FIG. 5) is transmitted to a seed-to-sale profile engine in block 510, which includes grower (cultivator) information, plant genetic information, reviews and user-generated feedback, and other information associated with the seed-to-sale process. In such, the user is provided with for each grower (see block 515) including their profile, related results, water regimen, nutrient regimen, as well as other product processing information. For example, the solvent data for a concentrate may be provided. In block 520, the plant genetic information of the seed-to-sale profile is stored including the parent plant genetics, as well as the certificate of analysis and laboratory results. In block 525, the user generated feedbacks are stored which includes reviews and similar user/patient/demographics.

In some embodiments, the water regimen and nutrient regimen may include the water composition (e.g., whether the water was provided with added nutrients), water schedule, nutrients used, a nutrient application schedule, and other associated information.

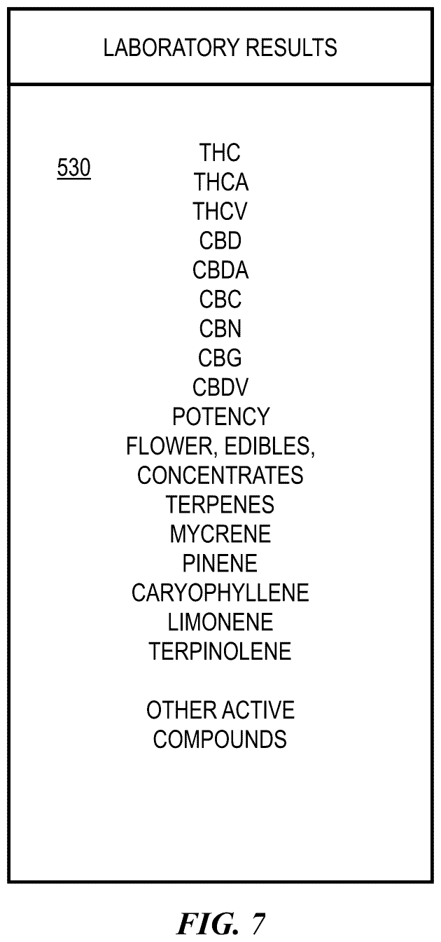

FIG. 7 illustrates block 530 of the flowchart. In block 530, laboratory results are stored which includes a plurality of information associated with each product. The laboratory results may include cannabinoid information (e.g., THC content, THCA content, THCV content, CBD content, CBDA content, CBC content, CBN content, CBG content, and CBDV content). The laboratory results may include potency tests, percentages by weight, or total milligrams present of each element of the laboratory test. Further, the product type may be indicated, such as whether the tested product was flower, an edible, a concentrate, a tincture, etc. Similarly, other metrics may be included such as the presence and/or concentrations of terpenes, mycyene, pinene, caryophyllene, limonene, terpinolene, among other active compounds.

In some embodiments, the terpenes and/or flavonoids may be displayed as a terpene profile and flavonoid profile respectively. For example, a terpene profile may include a list of each terpene, its associated scent(s) and flavor(s), and any associated benefits or effects.

In some embodiments, secondary, tertiary, or quaternary terpenes may be characterized by the system and displayed to the user.

In some embodiments, the product may be characterized as “THC-dominant,” “CBD-dominant,” or “Balanced THC/CBD.”

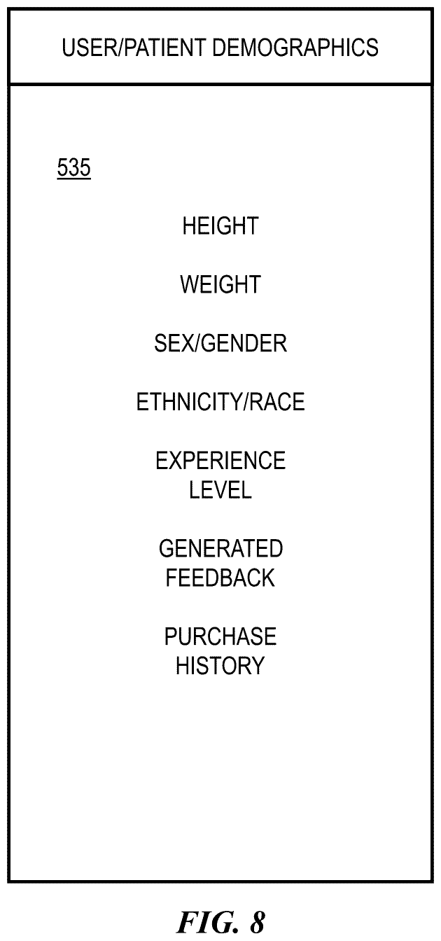

FIG. 8 illustrates block 535 of the flowchart. In block 535, user and patient demographics are stored including the user's height, weight, sex/gender, ethnicity/race, experience level, generated feedbacks, and purchase history. As shown in FIG. 8, information from the laboratory results (see block 530) and user/patient demographics (see block 535) is transmitted to the recommendation engine in block 540 wherein products can be recommended based on whether or not the user is a recreational user or medicinal patient. The recommendation engine can then suggest one or more products to be displayed to the user based on the user's preferences, and product information.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrase “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

What is claimed is:

1. A system for matching a cannabis user to a cannabis product, the system comprising:
at least one user computing device in operable connection with a user network;
an application server in operable communication with the user network, the application server configured to host an application system for providing a system for providing a recommendation engine to match a plurality of user information to a cannabis product which provides desired effects which match the plurality of user information, the application system having a user interface module for providing access to the application system through the user computing device, the user interface module in operable communication with: a user database and a product database, the user database storing user information and the product database for storing a plurality of product information, and the at least one user computing device operale to collect real-time usage metrics and biometric feedback;
a machine learning engine operable to process aggregated data to dynamically train personalized recommendation modules;
a lab data ingestion module to parse COA profile and convert the COA profiles to structured records;
a blockchain database operate to store product lineage, lab results, and seed-to-sale metadata; and
a seed-to-sale profile including the plurality of provider information, a plurality of plant genetics information, and one or more reviews, wherein the seed-to-sale profile is displayed via the user interface module,
wherein the recommendation engine receives the plurality of user information to match the user information to one or more cannabis products.

2. The system of claim 1, wherein biometric data includes at least one of: heart rate variability, sleep metrics, body temperature, blood oxygen saturation, and stress indicators collected from a wearable sensor.

3. The system of claim 1, wherein the federated learning protocol comprises local training of a neural network model on the user computing device and aggregation of encrypted model parameters to a central server.

4. The system of claim 1, wherein the machine learning model comprises a recurrent neural network (RNN) or long short-term memory (LSTM) model adapted to correlate time-series biometric and usage data.

5. The system of claim 1, wherein the lab data ingestion module performs optical character recognition and natural language processing to parse COAs submitted in PDF format.

6. The system of claim 1, wherein the product database includes strain-specific entries annotated with cannabinoid profiles, terpene profiles, product type, and contamination screening results.

7. A system for matching a cannabis user to a cannabis product, the system comprising:
at least one user computing device in operable connection with a user network;
an application server in operable communication with the user network, the application server configured to host an application system for providing a system for providing a recommendation engine to match a plurality of user information to a cannabis product which provides desired effects which match the plurality of user information, the application system having a user interface module for providing access to the application system through the user computing device, the user interface module in operable communication with: a user database and a product database, the user database storing user information and the product database for storing a plurality of product information;
a machine learning engine operable to process aggregated data to dynamically train personalized recommendation modules;
a lab data ingestion module to parse COA profile and convert the COA profiles to structured records;
a blockchain database operate to store product lineage, lab results, and seed-to-sale metadata;
a hashing module configured to generate a cryptographic hash of the extracted data;
a transparency interface operable to display to a user the product lineage, lab origin, and COA authenticity based on blockchain-verified entries; and
a seed-to-sale profile including the plurality of provider information, a plurality of plant genetics information, and one or more reviews, wherein the seed-to-sale profile is displayed via the user interface module;
a provider database to store a plurality of provider information including a product inventory associated with the provider,
wherein the recommendation engine receives the plurality of user information to match the user information to one or more cannabis products and to one or more providers, wherein matching the one or more providers are is determined by the product inventory and a provider location.

8. The system of claim 8, wherein the plurality of user information is comprised of at least one of the following:
at least one desired effect;
at least one symptom; and
at least one user type.

9. The system of claim 9, further comprising a plurality of plant genetics information includes at least one of the following:
one or more parent plant genetics;
at least one certificate of analysis; and
one or more laboratory results.

10. The system of claim 10, wherein the user information includes a plurality of user demographics including at least one of the following:
a height;
a weight;
a sex/gender;
an ethnicity/race;
an experience level;
one or more generated feedbacks; and
a purchase history.

11. The system of claim 11, wherein the product database includes a plurality of laboratory results to provide cannabinoid concentrations, a product type, a terpene profile, and a flavonoid profile.

12. The system of claim 12, wherein the recommendation engine recommends a dosage protocol based on the user information and the plurality of laboratory results.

13. The system of claim 2, wherein the phenotype data is derived from genomic analysis and includes at least one of: cannabis sensitivity markers, metabolic rate indicators, or anxiety predisposition variants.

14. The system of claim 2, wherein the recommendation includes a dosage protocol tailored to the user's body weight, tolerance history, and prior feedback on similar products.

15. The system of claim 2, further comprising the step of collecting real-time biometric data post-consumption to refine the prediction model using reinforcement learning.

16. A method for matching a cannabis user to a cannabis product, the method comprising the steps of:
receiving, from a user computing device, user information including at least biometric feedback and demographic data;
retrieving structured product data from a laboratory database including cannabinoid concentrations, terpene profiles, and lab validation metadata;
receiving phenotype data associated with the user;
executing a phenotype-to-strain prediction model to determine a recommended cannabis product for the user; and
providing a display of the recommended product, dosage, and route of administration based on the prediction model.

17. The method of claim 16, wherein the product includes a seed-to-sale profile.

18. The method of claim 17, wherein the seed-to-sale profile includes a plurality of provider information, a plurality of plant genetics, and one or more reviews.

19. The method of claim 18, wherein the plurality of user information is comprised of at least one of the following:
at least one desired effect;
at least one symptom; and
at least one user type.

20. The method of claim 19, wherein a plurality of plant genetics information includes at least one of the following:
one or more parent plant genetics;
at least one certificate of analysis; and
one or more laboratory results.
